(19) United States
(12) Patent Application Publication
(10) Pub. No.: US 2008/0114282 A1
(43) Pub. Date: May 15, 2008

(54) TRANSDERMAL DRUG DELIVERY SYSTEMS, DEVICES, AND METHODS USING INDUCTIVE POWER SUPPLIES

(75) Inventor: Darrick Carter, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: TRANSCU LTD., Singapore (SG)

(21) Appl. No.: 11/850,600

(22) Filed: Sep. 5, 2007

Related U.S. Application Data
(60) Provisional application No. 60/842,694, filed on Sep. 5, 2006.

(51) Int. Cl. A61N 1/30

(52) U.S. Cl. 604/20

(57) ABSTRACT

An iontophoresis device for providing transdermal delivery of one or more therapeutic active agents to a biological interface having an active electrode assembly, a counter electrode assembly, and an inductor electrically coupled to the active and the counter electrode assemblies. The inductor is operable to provide a voltage across at the active and the counter electrode elements in response to an applied varying electromagnetic field.

### CROSS-REFERENCE TO RELATED APPLICATION

This application claims the benefit under 35 U.S.C. § 119(e) of U.S. Provisional Patent Application No. 60/842,694 filed Sep. 5, 2006, the content of which is incorporated herein by reference in its entirety.

### BACKGROUND

1. Field

This disclosure generally relates to the field of iontophoresis and, more particularly, to systems, devices, and methods for delivering active agents such as analgesic drugs to a biological interface under the influence of an electromotive force.

2. Description of the Related Art

Iontophoresis employs an electromotive force and/or current to transfer an active agent (e.g., a charged substance, an ionized compound, an ionic drug, a therapeutic, a bioactive-agent, and the like), to a biological interface (e.g., skin, mucus membrane, and the like), by using a small electrical charge applied to an iontophoretic chamber containing a similarly charged active agent and/or its vehicle.

Iontophoresis devices typically include an active electrode assembly and a counter electrode assembly, each coupled to opposite poles or terminals of a power source, for example a chemical battery or an external power station connected to the iontophoresis device via electrical leads. Each electrode assembly typically includes a respective electrode element to apply an electromotive force and/or current. Such electrode elements often comprise a sacrificial element or compound, for example silver or silver chloride. The active agent may be either cationic or anionic, and the power source may be configured to apply the appropriate voltage polarity based on the polarity of the active agent. Iontophoresis may be advantageously used to enhance or control the delivery rate of the active agent. The active agent may be stored in a reservoir such as a cavity. See e.g., U.S. Pat. No. 5,395,310. Alternatively, the active agent may be stored in a reservoir such as a porous structure or a gel. An ion exchange membrane may be positioned to serve as a polarity selective barrier between the active agent reservoir and the biological interface. The membrane, typically only permeable with respect to one particular type of ion (e.g., a charged active agent), prevents the back flux of oppositely charged ions from the skin or mucous membrane.

Commercial acceptance of iontophoresis devices is dependent on a variety of factors, such as cost to manufacture, shelf life, stability during storage, efficiency and/or timeliness of active agent delivery, biological capability, and/or disposal issues. Commercial acceptance of iontophoresis devices is also dependent on their versatility and ease-of-use. Therefore, it may be desirable to have novel approaches for powering iontophoresis devices.

The present disclosure is directed to overcoming one or more of the shortcomings set forth above, and providing further related advantages.

### BRIEF SUMMARY

In one aspect, the present disclosure is directed to an iontophoresis device for providing transdermal delivery of one or more therapeutic active agents to a biological interface. The iontophoresis device includes an active electrode assembly, a counter electrode assembly, and an inductor. The active electrode assembly includes at least one active agent reservoir and at least one active electrode element operable to provide an electromotive force to drive the one or more active agents from the at least one active agent reservoir. The counter electrode assembly includes at least one counter electrode element. The inductor is electrically coupled to the active and the counter electrode elements for providing a voltage across at least the active and the counter electrode elements in response to a varying electromagnetic field applied to the inductor.

In another aspect, the present disclosure is directed to a system for delivering one or more active agents to a biological entity under the influence of an inductive power supply. The system includes an inductive power supply and an iontophoresis device. The inductive power supply includes a primary winding operable to produce a varying magnetic field. The iontophoresis device includes at least one active agent reservoir to store one or more active agents, an active electrode element operable to apply an electromotive force to the active agent reservoir, and a counter electrode element. The iontophoresis device further includes a secondary winding electrically coupled to the active and the counter electrode elements for providing a voltage across the active and counter electrode elements in response to the varying magnetic field of the inductive power supply.

In another aspect, the present disclosure is directed to a method of powering an iontophoretic delivery device. The method includes varying a current applied to a primary winding housed separately form the iontophoretic delivery device to generate a varying electromagnetic field, and positioning a secondary winding housed by the iontophoretic delivery device such that the secondary winding will be within the generated varying magnetic field.

In yet another aspect, the present disclosure is directed to a method of forming an inductively powered iontophoretic device. The method includes forming an inductor element on at least a first substrate having first and second opposing surfaces and electrically coupling the inductor element to an iontophoresis device. The iontophoresis device includes an active electrode assembly and a counter electrode assembly. The active electrode assembly includes at least one active agent reservoir and at least one active electrode element operable to provide an electromotive force to drive one or more active agents from the at least one active agent reservoir, and the counter electrode assembly includes at least one counter electrode element. The inductor element is operable to provide a voltage across at least the active and the counter electrode elements of the iontophoresis device in response to a varying electromagnetic field applied to the inductor from an external source.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements, and have been solely selected for ease of recognition in the drawings.

FIG. 1A is a block diagram of an iontophoresis device comprising active and counter electrode assemblies and an inductive power system according to one illustrated embodiment.

FIG. 1B is a block diagram of an expanded view of the inductive power system of FIGS. 1A and 2 according to another illustrated embodiment.

FIG. 2 is a block diagram of the iontophoresis device of FIG. 1A positioned on a biological interface, with the outer release liner removed to expose the active agent according to another illustrated embodiment.

FIG. 3A is a front top isometric view of an inductor according to one illustrated embodiment.

FIG. 3B is a top plan view of an inductor according to another illustrated embodiment.

FIG. 3C is a front top isometric view of an inductor according to another illustrated embodiment.

FIGS. 4A and 4B are front top isometric views of an inductor according to another illustrated embodiment.

FIG. 5 is a flow diagram of a method of powering an iontophoretic delivery device according to one illustrated embodiment.

FIG. 6 is a flow diagram of a method of forming an iontophoretic delivery device according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with iontophoresis devices including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “another embodiment” means that a particular referent feature, structure, or characteristic described in connection with the embodiment is included in at least one embodiment. Thus, the appearances of the phrases “in one embodiment,” or “in an embodiment,” or “another embodiment” in various places throughout this specification are not necessarily all referring to the same embodiment. Further more, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an iontophoresis device including “an inductor” includes a single inductor, or two or more inductors. It should also be noted that the term “or” is generally employed in its sense including “and/or” unless the content clearly dictates otherwise.

As used herein the term “membrane” means a boundary, layer, barrier, or material, which may, or may not be permeable. The term “membrane” may further refer to an interface. Unless specified otherwise, membranes may take the form of a solid, a liquid, or a gel, and may or may not have a distinct lattice, non-cross-linked structure, or cross-linked structure.

As used herein the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH, and ACS, also from Tokuyama Co., Ltd.

As used herein and in the claims, the term “bipolar membrane” means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two-film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules at a first rate, and some other molecules at a second rate different from the first. In yet further embodiments, the “semi-permeable membrane” may take the form of a selectively permeable membrane allowing only certain selective molecules to pass through it.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three-dimensional network, a colloidal suspension of a liquid in a solid, a semi-solid, a cross-linked gel, a non-cross-linked gel, a jelly-like state, and the like. In some embodiments, the gel matrix may result from a three-dimensional network of entangled macromolecules (e.g., cylindrical micelles). In some embodiments, a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

As used herein and in the claims, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

As used herein and in the claims, the term “active agent” refers to a compound, molecule, or treatment that elicits a biological response from any host, animal, vertebrate, or invertebrate, including, for example fish, mammals, amphibians, reptiles, birds, and humans. Examples of active agents include therapeutic agents, pharmaceutical agents, pharmaceuticals (e.g., a drug, a therapeutic compound, pharmaceutical salts, and the like) non-pharmaceuticals (e.g., a cosmetic substance, and the like), a vaccine, an immunological agent, a local or general anesthetic or painkiller, an antigen or a protein or peptide such as insulin, a chemotherapy agent, and an anti-tumor agent. In some embodiments, the term “active agent” refers to the active agent as well as to its pharmacologically active salts, pharmaceutically acceptable salts, prodrugs, metabolites, analogs, and the like. In some further embodiment, the active agent includes at least one ionic, cationic, ionizeable and/or neutral therapeutic drug and/or pharmaceutically acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. Other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH. The term “active agent” may also refer to electrically neutral agents, molecules, or compounds capable of being delivered via electro-osmotic flow. The electrically neutral agents are typically carried by the flow of, for example, a solvent during electrophoresis. Selection of the suitable active agents is therefore within the knowledge of one skilled in the relevant art.

In some embodiments, one or more active agents may be selected from analgesics, anesthetics, anesthetics vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of such active agents include lidocaine, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of agents include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (Novocaine®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

As used herein and in the claims, the term “subject” generally refers to any host, animal, vertebrate, or invertebrate, and includes fish, mammals, amphibians, reptiles, birds, and particularly humans.

The headings provided herein are for convenience only and do not interpret the scope or meaning of the embodiments.

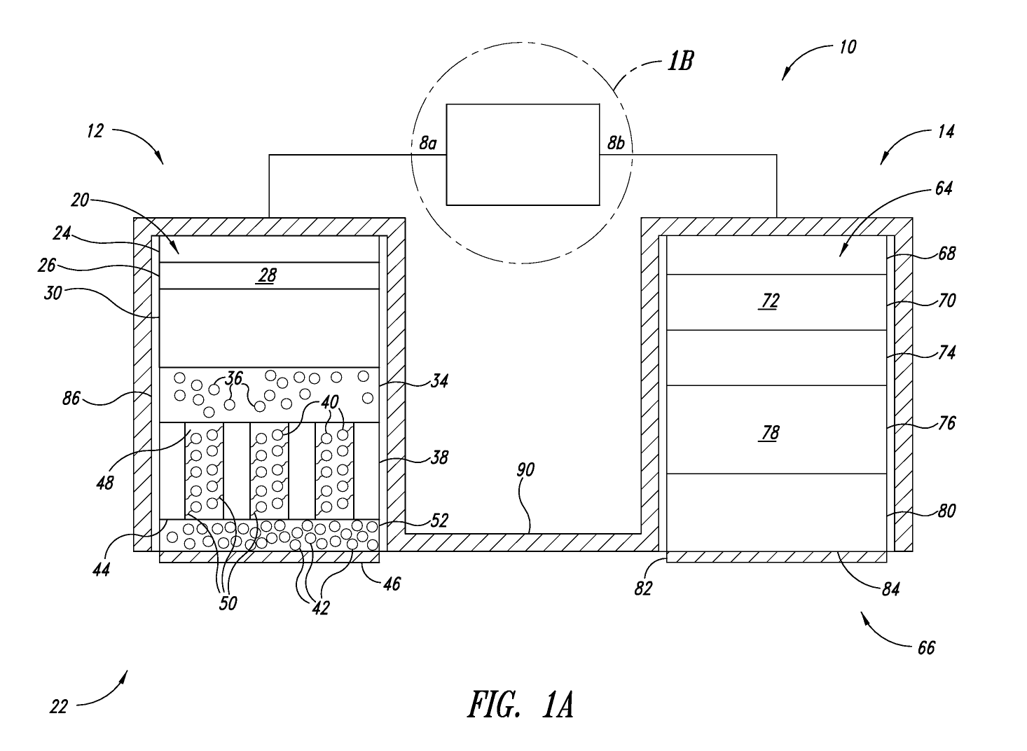

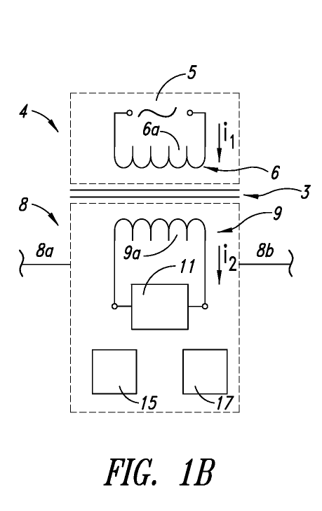

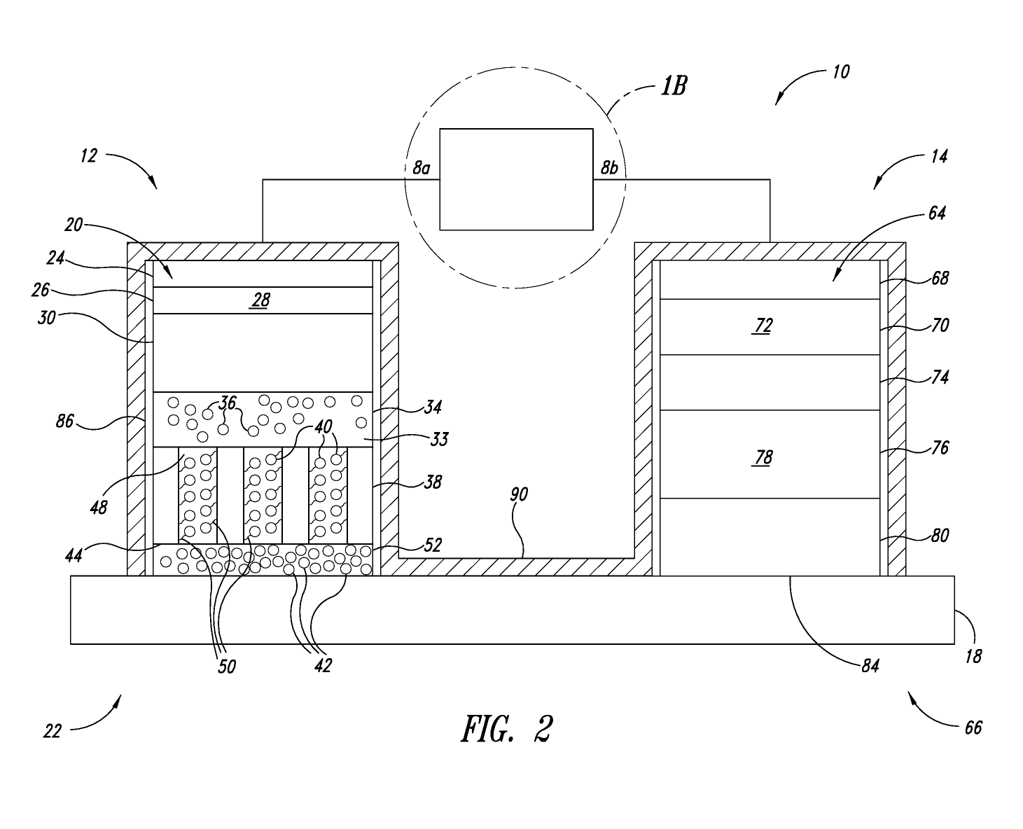

FIGS. 1A, 1B, and 2 show an exemplary system 2 for delivering one or more active agents to a biological entity under the influence of an inductive power supply. The system 2 includes an inductive power supply 4 including and inductor 6, and an iontophoresis device 10 including an inductor 9.

The inductive power supply 4 is operable to transfer energy, via inductive coupling, from one component to another through a shared magnetic field 3. A change in current flow (i₁) through one component may induce a current flow (i₂) in the other component. The transfer of energy results in part from the mutual inductance between the components. For example, the inductive power supply 4 is operable to transfer energy, via inductive coupling, from a primary inductor 6 to a secondary inductor 9 through a shared magnetic field 3.

In an embodiment, the inductive power supply 4 may include one or more inductors 6 operable to produce one or more varying magnetic fields 3. Examples of inductor 6 include a coil, a winding, a primary coil, a primary winding, an inductive coil, a primary inductor, and the like. In an embodiment, the inductor 6 may take the form of a planar inductor. In another embodiment, the inductive power supply 4 may include an inductor 6 in the form of a primary winding 6a operable to produce a varying magnetic field 3. A winding 6a may include one or more complete turns of a conductive material in a coil, and may comprise one or more layers. Examples of suitable conductive materials include conductive polymers, metallic materials, copper, gold, silver, copper coated with silver or tin, aluminum, and/or alloys. In some embodiments, the winding 6a may comprise, for example, solid wires, including, for example, flat wires, strands, twisted strands, sheets, and the like.

The inductive power supply 4 may further be operable to provide at least one of an alternating current 5 or a pulsed direct current (not shown) to the primary winding 6a. In response to the alternating current 5 or a pulsed direct current, the one or more windings 6a of the inductive power supply 4 may produce one or more varying magnetic fields 3.

A “duty cycle” refers to a ratio of a pulse signal duration relative to a pulse signal period. For example, a pulse signal duration of 10 μs and a pulse signal period of 50 μs, correspond to a duty cycle of 0.2. In an embodiment, the inductive power supply 4 is operable to manage a duty cycle associated with delivering a therapeutically effective amount of one or more active agents 36, 40, 42.

The iontophoresis device 10 includes an active electrode assembly 12 and counter electrode assembly 14. The iontophoresis device 10 further includes a power source 8, including one or more inductors 9 electrically coupled to the active and counter electrode assemblies 12, 14. The inductor 9 is operable to provide a voltage across the active and counter electrode assemblies 12, 14, in response to the varying magnetic field 3 of the inductive power supply 4. In an embodiment the inductor 9 may include one or more secondary windings 9a electrically coupled to the active and counter electrode assemblies 12, 14, for providing a voltage across the active and counter electrode assemblies 12, 14, in response to the varying magnetic field 3 of the inductive power supply 4. The iontophoresis device 10 is operable to supply one or more active agents 36, 40, 42 contained in the active electrode assembly 12 to a biological interface 18 (e.g., a portion of a skin or mucous membrane) via iontophoresis.

The one or more secondary windings 9a may include one or more complete turns of a conductive material in a coil, and may comprise one or more layers. Examples of suitable conductive materials include conductive polymers, metallic materials, copper, gold, silver, copper coated with silver or tin, aluminum, and/or alloys. In some embodiments, the one or more secondary windings 9a may comprise, for example, solid wires, including, for example, flat wires, strands, twisted strands, sheets, and the like. In other embodiments, the one or more secondary windings 9a may comprise one or more laminates that include windings to form an inductor.

In an embodiment, the inductive power supply 4 and the power source 8 may comprise a two-part transformer having a primary coil included in the inductive power supply 4, and one or more secondary coils included in the iontophoresis device 10. Placing the secondary coil proximate to the varying magnetic field 3 generated by the inductive power supply 4, including the primary coil, induces a current in the secondary coil. The induced current can in turn supply power to the iontophoresis device 10.

The iontophoresis device 10 may also include discrete and/or integrated circuit elements 15, 17 to control the voltage, current and/or power delivered to the electrode assemblies 12, 14. For example, the iontophoresis device 10 may include a diode to provide a constant current to the electrode elements 24, 68. In some embodiments, the iontophoresis device 10 may include a rectifying circuit to provide a direct current voltage and/or a voltage/current regulator. In other embodiments, the iontophoresis device 10 may include a circuit operable to sinks and sources voltage to maintain a steady state operation of the iontophoresis device 10.

The power source 8 may further include a rechargeable power source 11 electrically coupled to the active and counter electrode assemblies 12, 14, and electrically coupled in parallel with the inductor 9 to receive a charge thereby. Examples of the inductor 9 include a coil, a winding, a secondary coil, a secondary winding, an inductive coil, a secondary inductor, and the like. In an embodiment, the inductor 9 may take the form of a planar inductor.

In an embodiment, the power source 8 may include at least one of a chemical battery cell, super- or ultra-capacitor, a fuel cell, a secondary cell, a thin film secondary cell, a button cell, a lithium ion cell, zinc air cell, a nickel metal hydride cell, and the like. In certain embodiments, the rechargeable power source sinks and sources voltage to maintain a steady state operation of the iontophoresis device. The power source 8 may, for example, provide a voltage of 12.8 V DC, with tolerance of 0.8 V DC, and a current of 0.3 mA. The power source 8 may be selectively electrically coupled to the active and counter electrode assemblies 12, 14 via a control circuit 15, for example, via carbon fiber ribbons.

The active electrode assembly 12 of the iontophoresis device 10 may further comprise, from an interior 20 to an exterior 22 of the active electrode assembly 12: an active electrode element 24, an electrolyte reservoir 26 storing an electrolyte 28, an inner ion selective membrane 30, an inner active agent reservoir 34, storing one or more active agents 36, an optional outermost ion selective membrane 38 that optionally caches additional active agents 40, an optional further active agent 42 carried by an outer surface 44 of the outermost ion selective membrane 38, and an optional outer release liner 46. The active electrode assembly 12 may further comprise an optional inner sealing liner (not shown) between two layers of the active electrode assembly 12, for example, between the inner ion selective membrane 30 and the inner active agent reservoir 34. The inner sealing liner, if present, would be removed prior to application of the iontophoretic device to the biological surface 18. Each of the above elements or structures will be discussed in detail below.

The active electrode element 24 is electrically coupled to a first pole 8a of the power source 8 and positioned in the active electrode assembly 12 to apply an electromotive force to transport the active agent 36, 40, 42 via various other components of the active electrode assembly 12.

The active electrode element 24 may take a variety of forms. In one embodiment, the device may advantageously employ a carbon-based active electrode element 24. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004. The carbon-based electrodes are inert electrodes in that they do not themselves undergo or participate in electrochemical reactions. Thus, an inert electrode distributes current without being eroded or depleted, and conducts current through electrolysis of water (i.e., generating ions by either reduction or oxidation of water). Additional examples of inert electrodes include stainless steel, gold, platinum, or graphite.

Alternatively, an active electrode of sacrificial conductive material, such as a chemical compound or amalgam, may also be used. A sacrificial electrode does not cause electrolysis of water, but would itself be oxidized or reduced. Typically, for an anode a metal/metal salt may be employed. In such case, the metal would oxidize to metal ions, which would then be precipitated as an insoluble salt. An example of such anode includes an Ag/AgCl electrode. The reverse reaction takes place at the cathode in which the metal ion is reduced and the corresponding anion is released from the surface of the electrode.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, or a membrane with pores, cavities, or interstices, particularly where the electrolyte 28 is a liquid.

In one embodiment, the electrolyte 28 comprises ionic or ionizable components in an aqueous medium, which can act to conduct current towards or away from the active electrode element. Suitable electrolytes include, for example, aqueous solutions of salts. Preferably, the electrolyte 28 includes salts of physiological ions, such as, sodium, potassium, chloride, and phosphate.

Once an electrical potential is applied, when an inert electrode element is in use, water is electrolyzed at both the active and counter electrode assemblies. In certain embodiments, such as when the active electrode assembly is an anode, water is oxidized. As a result, oxygen is removed from water while protons (H⁺) are produced. In one embodiment, the electrolyte 28 may further comprise an anti-oxidant to inhibit the formation of oxygen gas bubbles in order to enhance efficiency and/or increase delivery rates. Examples of biologically compatible anti-oxidants include, but are not limited to ascorbic acid (vitamin C), tocopherol (vitamin E), or sodium citrate.

As noted above, the electrolyte 28 may take the form of an aqueous solution housed within a reservoir 26, or may take the form of a dispersion in a hydrogel or hydrophilic polymer capable of retaining a substantial amount of water. For instance, a suitable electrolyte may take the form of a solution of 0.5 M disodium fumarate: 0.5 M polyacrylic acid: 0.15 M anti-oxidant.

The inner ion selective membrane 30 is generally positioned to separate the electrolyte 28 and the inner active agent reservoir 34, if such a membrane is included within the device. The inner ion selective membrane 30 may take the form of a charge selective membrane. For example, when the active agent 36, 40, 42 comprises a cationic active agent, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na⁺) ions from the electrolyte 28, thereby increasing the transfer rate and/or biological compatibility of the iontophoresis device 10.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36. For example, the inner active agent reservoir 34 may take the form of a pouch or other receptacle, a membrane with pores, cavities, or interstices, particularly where the active agent 36 is a liquid. The inner active agent reservoir 34 further may comprise a gel matrix.

Optionally, an outermost ion selective membrane 38 is positioned generally opposed across the active electrode assembly 12 from the active electrode element 24. The outermost membrane 38 may, as in the embodiment illustrated in FIGS. 1A and 2, take the form of an ion exchange membrane having pores 48 (only one called out in FIGS. 1A and 2 for sake of clarity of illustration) of the ion selective membrane 38 including ion exchange material or groups 50 (only three called out in FIGS. 1A and 2 for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., lidocaine), the outermost ion selective membrane 38 may take the form of a cation exchange membrane, thus allowing the passage of the cationic active agent while blocking the back flux of the anions present in the biological interface, such as skin.

The outermost ion selective membrane 38 may optionally cache active agent 40. Without being limited by theory, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of semi-permeable or microporous membrane that is selective by size. In some embodiments, such a semi-permeable membrane may advantageously cache active agent 40, for example by employing the removably releasable outer release liner 46 to retain the active agent 40 until the outer release liner 46 is removed prior to use.

The outermost ion selective membrane 38 may be optionally preloaded with the additional active agent 40, such as ionized or ionizable drugs or therapeutic agents and/or polarized or polarizable drugs or therapeutic agents. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically depositing, vapor depositioning, and/or otherwise. In some embodiments, the further active agent 42 may sufficiently cover the outer surface 44 and/or be of sufficient thickness to form a distinct layer 52. In other embodiments, the further active agent 42 may not be sufficient in volume, thickness or coverage as to constitute a layer in a conventional sense of such term.

The active agent 42 may be deposited in a variety of highly concentrated forms such as, for example, solid form, nearly saturated solution form, or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

The outer release liner 46 may generally be positioned overlying or covering further active agent 42 carried by the outer surface 44 of the outermost ion selective membrane 38. The outer release liner 46 may protect the further active agent 42 and/or outermost ion selective membrane 38 during storage, prior to application of an electromotive force or current. The outer release liner 46 may be a selectively releasable liner made of waterproof material, such as release liners commonly associated with pressure sensitive adhesives. Note that the outer release liner 46 is shown in place in FIG. 1A and removed in FIG. 2.

An interface-coupling medium (not shown) may be employed between the electrode assembly and the biological interface 18. The interface coupling medium may take the form of, for example, an adhesive and/or gel. The gel may take the form of, for example, a hydrating gel. Selection of suitable bioadhesive gels is within the knowledge of one skilled in the relevant art.

In the embodiment illustrated in FIGS. 1A and 2, the counter electrode assembly 14 comprises, from an interior 64 to an exterior 66 of the counter electrode assembly 14: a counter electrode element 68, an electrolyte reservoir 70 storing an electrolyte 72, an inner ion selective membrane 74, an optional buffer reservoir 76 storing buffer material 78, an optional outermost ion selective membrane 80, and an optional outer release liner 82.

The counter electrode element 68 is electrically coupleable via a second pole 8b to the power source 8, the second pole 8b having an opposite polarity to the first pole 8a. In one embodiment, the counter electrode element 68 is an inert electrode. For example, the counter electrode element 68 may take the form of the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities, or interstices, particularly where the electrolyte 72 is a liquid.

The electrolyte 72 is generally positioned between the counter electrode element 68 and the outermost ion selective membrane 80, proximate the counter electrode element 68. As described above, the electrolyte 72 may provide ions or donate charges to prevent or inhibit the formation of gas bubbles (e.g., hydrogen or oxygen, depending on the polarity of the electrode) on the counter electrode element 68 and may prevent or inhibit the formation of acids or bases or neutralize the same, which may enhance efficiency and/or reduce the potential for irritation of the biological interface 18.

The inner ion selective membrane 74 may be positioned between the electrolyte 72 and the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxy (OH⁻) or chloride (Cl⁻) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane, or a gel.

The buffer material 78 may supply ions for transfer through the outermost ion selective membrane 42 to the biological interface 18. Consequently, the buffer material 78 may comprise, for example, a salt (e.g., NaCl).

The outermost ion selective membrane 80 of the counter electrode assembly 14 may take a variety of forms. For example, the outermost ion selective membrane 80 may take the form of a charge selective ion exchange membrane. Typically, the outermost ion selective membrane 80 of the counter electrode assembly 14 is selective to ions with a charge or polarity opposite to that of the outermost ion selective membrane 38 of the active electrode assembly 12. The outermost ion selective membrane 80 is therefore an anion exchange membrane, which substantially passes anions and blocks cations, thereby prevents the back flux of the cations from the biological interface. Examples of suitable ion exchange membranes include the previously discussed membranes.

Alternatively, the outermost ion selective membrane 80 may take the form of a semi-permeable membrane that substantially passes and/or blocks ions based on size or molecular weight of the ion.

The outer release liner 82 may generally be positioned overlying or covering an outer surface 84 of the outermost ion selective membrane 80. Note that the outer release liner 82 is shown in place in FIG. 1A and removed in FIG. 2. The outer release liner 82 may protect the outermost ion selective membrane 80 during storage, prior to application of an electromotive force or current. The outer release liner 82 may be a selectively releasable liner made of waterproof material, such as release liners commonly associated with pressure sensitive adhesives. In some embodiments, the outer release liner 82 may be coextensive with the outer release liner 46 of the active electrode assembly 12.

The iontophoresis device 10 may further comprise an inert molding material 86 adjacent exposed sides of the various other structures forming the active and counter electrode assemblies 12, 14. The molding material 86 may advantageously provide environmental protection to the various structures of the active and counter electrode assemblies 12, 14. Enveloping the active and counter electrode assemblies 12, 14 is a housing material 90.

As best seen in FIG. 2, the active and counter electrode assemblies 12, 14 are positioned on the biological interface 18. Positioning on the biological interface may close the circuit, allowing electromotive force to be applied and/or current to flow from one pole 8a of the power source 8 to the other pole 8b, via the active electrode assembly, biological interface 18 and counter electrode assembly 14.

In use, the outermost active electrode ion selective membrane 38 may be placed directly in contact with the biological interface 18. Alternatively, an interface-coupling medium (not shown) may be employed between the outermost active electrode ion selective membrane 22 and the biological interface 18. The interface-coupling medium may take the form of, for example, an adhesive and/or gel. The gel may take the form of, for example, a hydrating gel or a hydrogel. If used, the interface-coupling medium should be permeable by the active agent 36, 40, 42.

As suggested above, the one or more active agents 36, 40, 42 may take the form of one or more ionic, cationic, anionic, ionizeable, and/or neutral drugs or other therapeutic agents. Consequently, the poles or terminals of the power source 8 and the selectivity of the outermost ion selective membranes 38, 80 and inner ion selective membranes 30, 74 are selected accordingly.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small more highly charged active agents under the influence of these forces. These higher molecular active agents may thus be carried through the biological interface into the underlying tissues primarily via electroosmotic solvent flow. In certain embodiments, the high molecular weight polyelectrolytic active agents may be proteins, polypeptides or nucleic acids. In other embodiments, the active agent may be mixed with another agent to form a complex capable of being transported across the biological interface via one of the motive methods described above.

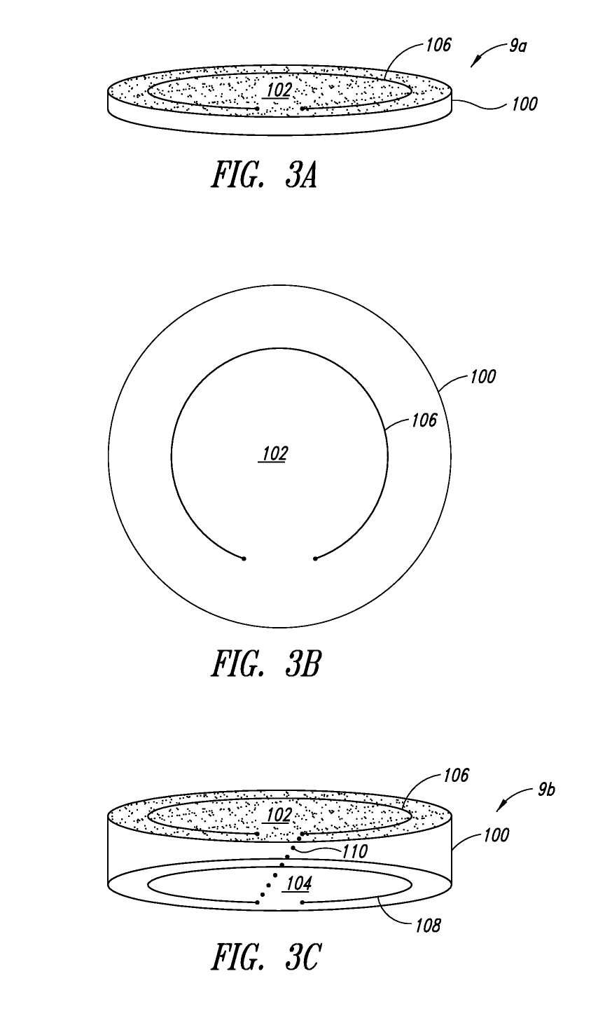

As shown in FIGS. 3A and 3B, the iontophoresis device 10 (FIGS. 1A and 1B) may include at least one inductor 9a comprising a substrate 100 having at least a first surface 102, and a second surface 104 opposed to the first surface 102. The first surface 102 may include an inductor 9a formed in part by a conductive trace 106 carried by the first surface 102 of the at least one substrate 100. In an embodiment, the inductor 9a may include a secondary winding in the form of a conductive trace 106 carried by the first surface 102. In certain embodiments, the conductive trace 106 may take the form of a geometric pattern including polygonal loops, square loops, circular loops (as shown), spiral patterns, concentric geometric shape patterns, and the like. Varying the winding geometry, the number of windings, the thickness of the conductive trace 106, the material composition of the conductive trace, and the like, may change the inductive properties of inductor 9a.

As shown in FIG. 3C, the iontophoresis device 10 (FIGS. 1A and 1B) may include at least one inductor 9b comprising a substrate 100 having at least a first surface 102, and a second surface 104 opposed to the first surface 102. The first and second surfaces 102, 104 may include an inductor 9b formed in part by a conductive trace 106 carried by the first surface 102 that is electrically coupled via electric connection 110 to a conductive trace 108 carried by the second surface 104 of the substrate 100. In an embodiment, the substrate 100 comprises an insulating or dielectric material, and the traces 106, 108 comprise a conductive material. In another embodiment, the conductive traces 106, 108 may comprise a conductive material and may include an electrically insulating layer or covering.

In certain embodiments, the inductor 9 may take the form of conductive traces 106, 108 deposited, etched, or otherwise applied to the substrate 100 and electrically configured to form a resonance circuit that is resonant at a particular resonance frequency.

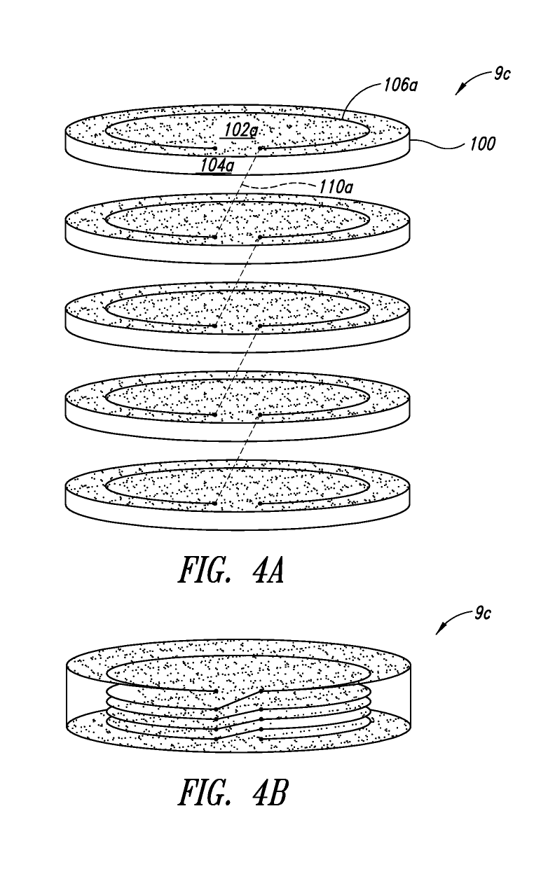

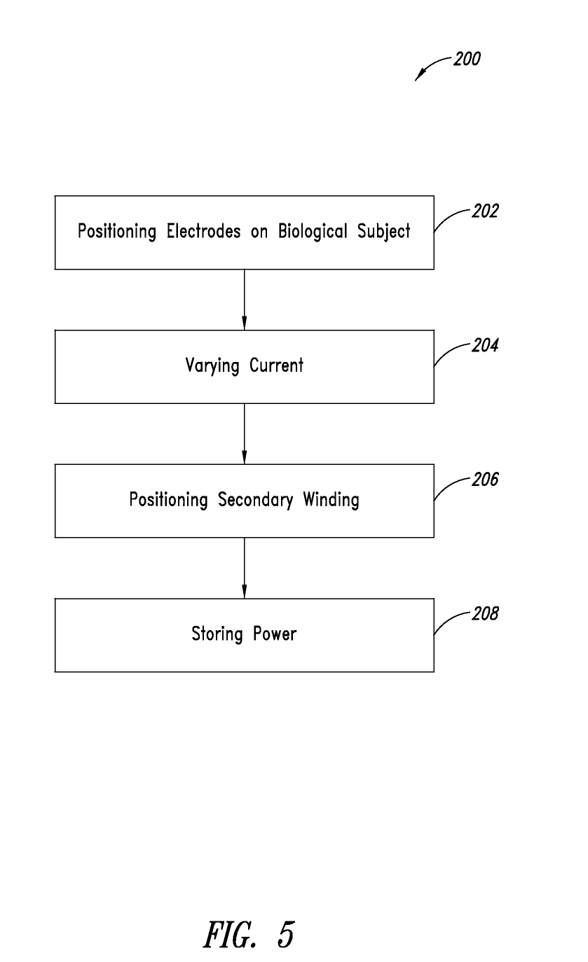

FIGS. 4A and 4B show an exemplary inductor 9c for an iontophoresis device 10 (FIGS. 1A and 1B) comprising multiple windings, turns, or coils. The inductor 9c may include two or more substrates 100a having at least a first surface 102a, and a second surface 104a opposed to the first surface 102a. The first surface 102a may include an inductor winding formed in part by a conductive trace 106a carried by the first surface 102a of the at least one substrate 100a. Each conductive trace 106a is electrically coupleable to an adjacent conductive trace 106a via an electrical coupling 110a to form the inductor 9c. In an embodiment, the inductor 9c may take the form of a laminate including at least two windings, turns, or coils. In another embodiment, adjacent electrically coupled conductive traces 106a are separated by a contiguous insulating substrate 100a to form a multi-winding inductor. In the example shown in FIG. 4B, the exemplary inductor 9c includes a multi-winding laminate.

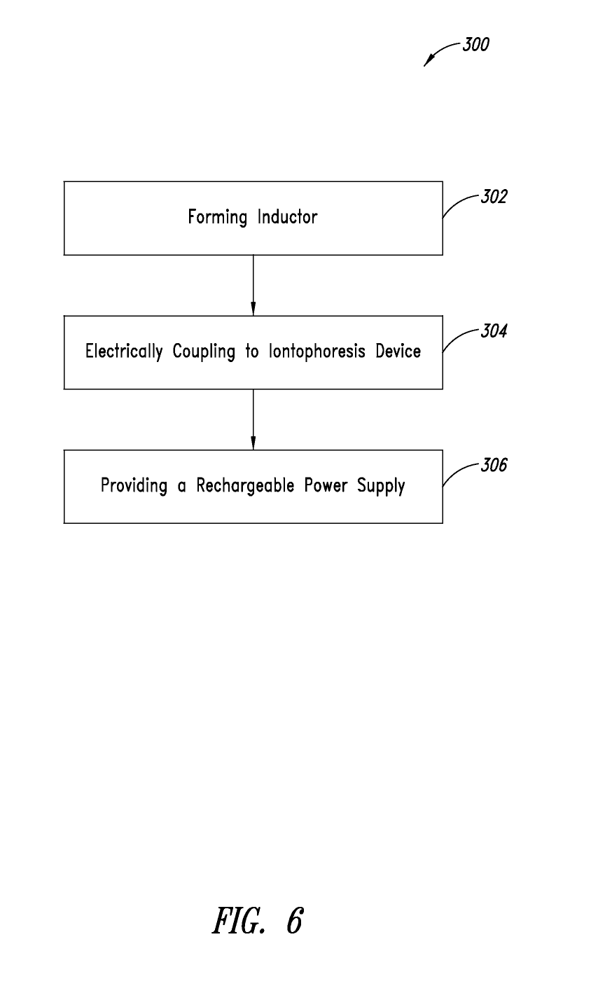

FIG. 5 shows an exemplary method 200 of powering iontophoretic delivery devices.

At 202, the method 200 may include positioning an active electrode and a counter electrode of an iontophoretic delivery device on a biological subject.

At 204, the method 200 includes applying a varying a current to a primary winding to generate a varying electromagnetic field. In an embodiment, varying the current applied to the primary winding may include varying the current according to a delivery profile. In another embodiment, varying the current applied to the primary winding may include varying the current according to a dosing and delivery profile to provide optimal dosing and delivery of one or more therapeutic agents. In another embodiment, varying the current applied to the primary winding may include varying the current to achieve delivery of a predetermined dosage necessary to achieve a therapeutic effect. In another embodiment, varying the current applied to the primary winding may include varying the current according to a delivery profile based on the one or more active agents. In yet another embodiment, varying the current applied to the primary winding may include varying the current according to a delivery profile based on at least one parameter indicative of a physical feature of the biological subject.

At 206, a secondary winding of the iontophoretic delivery device is position such that the secondary winding will be within the varying magnetic field when generated.

At 208, the method 200 may further include storing power to a rechargeable power supply. In some embodiments, the method 200 may further include positioning an active electrode and a counter electrode of the iontophoretic delivery device on a biological subject after storing power to the rechargeable power supply before varying the current applied to the primary winding to generate the varying electromagnetic field such that active agent is supplied to the biological entity in response to stored power.

In some embodiments, the method 200 may further include positioning an active electrode and a counter electrode of the iontophoretic delivery device on a biological subject before varying the current applied to the primary winding to generate the varying electromagnetic field such that active agent is supplied to the biological entity in response to varying the current.

FIG. 6 shows an exemplary method 300 of forming an inductively powered iontophoretic device.

At 302, the method 300 includes forming an inductor element on a substrate having a first surface and a second surface opposing the first surface. Well know lithographic techniques, for example, can be use to form an inductor element, or conductive trace layout, onto the first surface of the substrate. The lithographic process for forming the inductor element may include, for example, applying a resist film (e.g., spin-coating a photoresist film) onto the substrate, exposing the resist with an image of the inductor element layout (e.g., the geometric pattern of one or more conductive traces), heat treating the resist, developing the resist, transferring the layout onto the substrate, and removing the remaining resist. Transferring the layout onto the substrate may further include using techniques like subtractive transfer, etching, additive transfer, selective deposition, impurity doping, ion implantation, and the like.

In an embodiment, forming the inductor element on the substrate may include depositing a conductive trace, operable to provide a voltage across at least the active and the counter electrode elements in response to a varying electromagnetic field applied to the conductive trace, on at least the first surface of the substrate.

In an embodiment, at 302, the method 300 may includes forming an inductor element on a first substrate having a first surface and a second surface opposing the first surface, and forming an inductor element on at least a second substrate having a first surface and a second surface opposing the first surface. Forming the inductor element on the first and the at least second substrates may include depositing a first conductive trace on the first surface of the first substrate, depositing a second conductive trace on the first surface of the at least second substrate, and forming a laminate comprising the first and the at least second substrates. The first and the second conductive traces are electrically coupled to form a multi-loop inductor, and the electrically coupled first and the second conductive traces are operable to provide a voltage across at least the active and the counter electrode elements in response to a varying electromagnetic field, from an external source, applied to the first and the second conductive traces.

In an embodiment, at 302, forming the inductor element on the substrate may include forming a photoresist mask for patterning the conductive trace on the first surface of the substrate; and etching the conductive trace on the first surface of the substrate.

At 304, the method 300 includes electrically coupling the inductor element to an iontophoresis device comprising an active electrode assembly and a counter electrode assembly, the active electrode assembly including at least one active agent reservoir and at least one active electrode element operable to provide an electromotive force to drive an active agent from the at least one active agent reservoir, the counter electrode assembly including at least one counter electrode element. The inductor element is operable to provide a voltage across at least the active and the counter electrode elements in response to a varying electromagnetic field applied to the inductor.

At 306, the method 300 may include providing a rechargeable power supply electrically coupled to the inductor. In an embodiment, the rechargeable power supply may be operable to store power provided by the inductor in response to an applied varying electromagnetic field.

The above description of illustrated embodiments, including what is described in the Abstract, is not intended to be exhaustive or to limit the claims to the precise forms disclosed. Although specific embodiments and examples are described herein for illustrative purposes, various equivalent modifications can be made without departing from the spirit and scope of the disclosure, as will be recognized by those skilled in the relevant art. The teachings provided herein can be applied to other agent delivery systems and devices, not necessarily the exemplary iontophoresis active agent system and devices generally described above. For instance, some embodiments may include additional structure. For example, some embodiment may include a control circuit or subsystem to control a voltage, current or power applied to the active and counter electrode elements 20, 68. Also for example, some embodiments may include an interface layer interposed between the outermost active electrode ion selective membrane 22 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240,995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

The iontophoresis device discussed above may advantageously be combined with other microstructures, for example, microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedle arrays, comprising a plurality of microneedles, may be arranged in a variety of configurations, for example rectangular or circular. Microneedles and microneedle arrays may be manufactured from a variety of materials, including silicon; silicon dioxide; molded plastic materials, including biodegradable or non-biodegradable polymers; ceramics; and metals. Microneedles, either individually or in arrays, may be used to dispense or sample fluids through the hollow apertures, through the solid permeable or semi-permeable materials, or via the external grooves. Microneedle devices are used, for example, to deliver a variety of compounds and compositions to the living body via a biological interface, such as skin or mucous membrane. In certain embodiments, the compounds and drugs may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight active agents, such as those comprising proteins, peptides and/or nucleic acids, and corresponding compositions thereof. In certain embodiments, for example wherein the fluid is an ionic solution, microneedle(s) or microneedle array(s) can provide electrical continuity between a power source and the tip of the microneedle(s). Microneedle(s) or microneedle array(s) may be used advantageously to deliver or sample compounds or compositions by iontophoretic methods, as disclosed herein.

Accordingly, in certain embodiments, for example, a plurality of microneedles in an array may advantageously be formed on an outermost biological interface-contacting surface of an iontophoresis device. Compounds or compositions delivered or sampled by such a device may comprise, for example, high-molecular weight active agents, such as proteins, peptides, and/or nucleic acids.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir having a drug solution that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the voltage source; a second electrolyte holding part that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the voltage source; a first electrolyte reservoir having an electrolyte that is in contact with the first electrode member and to which is applied a voltage via the first electrode member; a first anion-exchange membrane that is placed on the forward surface of the first electrolyte holding part; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the voltage source; a second electrolyte holding part having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte holding part and the cation-exchange membrane; a second anion-exchange membrane placed against the forward surface of the third electrolyte reservoir; and a second cover or container that accommodates these members.

Certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; and 6,939,311. Some or all of the teachings therein may be applied to microneedle devices, their manufacture, and their use in iontophoretic applications.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: U.S. Provisional Patent Application No. 60/842,694, filed Sep. 5, 2006; Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No WO03037425; U.S. patent publication No. 2005-0070840 A1, published Mar. 31, 2005; Japanese patent application 2004/317317, filed Oct. 29, 2004; U.S. provisional patent application Ser. No. 60/627,952, filed Nov. 16, 2004; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; and Japanese patent application Serial No. 2005-081220, filed Mar. 22, 2005.

As one of skill in the art would readily appreciate, the present disclosure comprises methods of treating a subject by any of the compositions and/or methods described herein.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs, or other structures. Still other embodiments may employ additional ones of the membranes, reservoirs, and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

What is claimed is:

1. A system for delivering one or more active agents to a biological entity under the influence of an inductive power supply, comprising:
an inductive power supply including a primary winding operable to produce a varying magnetic field; and
an iontophoresis device including at least one active agent reservoir to store the one or more active agents, an active electrode element operable to apply an electromotive force to the active agent reservoir, a counter electrode element, and a secondary winding electrically coupled to the active and the counter electrode elements for providing a voltage across the active and counter electrode elements in response to the varying magnetic field of the inductive power supply;
wherein the iontophoresis device is physically distinct from the inductive power supply.

2. The system of claim 1 wherein the inductive power supply is operable to provide at least one of an alternating current or a pulsed direct current to the primary winding.

3. The system of claim 1 wherein the iontophoresis device includes a rechargeable power source electrically coupled to the active and counter electrode elements, and electrically coupled in parallel with the secondary winding to receive a charge thereby.

4. The system of claim 3 wherein the rechargeable power source sinks and sources voltage to maintain a steady state operation of the iontophoresis device.

5. The iontophoresis device of claim 16 wherein the rechargeable power source comprises at least one of a chemical battery cell, super- or ultra-capacitor, a fuel cell, a secondary cell, a thin film secondary cell, a button cell, a lithium ion cell, zinc air cell, and a nickel metal hydride cell.

6. The system of claim 1 wherein the inductive power supply is operable to manage a duty cycle associated with delivering a therapeutically effective amount of the one or more active agents.

7. The system of claim 1 wherein the inductive power supply is operable to provide at least one of an alternating current or a pulsed direct current to the primary winding with a duty cycle based on a delivery profile defined for at least one of the one or more active agents or the biological entity.

8. A method of powering an iontophoretic delivery device, the method comprising:
varying a current applied to a primary winding housed separately form the iontophoretic delivery device to generate a varying electromagnetic field; and
positioning a secondary winding housed by the iontophoretic delivery device such that the secondary winding will be within the varying magnetic field when generated.

9. The method of claim 8, further comprising:
positioning an active electrode and a counter electrode of the iontophoretic delivery device on a biological subject.

10. The method of claim 8, further comprising:
positioning an active electrode and a counter electrode of the iontophoretic delivery device on a biological subject before varying the current applied to the primary winding to generate the varying electromagnetic field such that active agent is supplied to the biological entity in response to varying the current.

11. The method of claim 8 wherein varying the current applied to the primary winding includes varying the current according to a delivery profile.

12. The method of claim 8 wherein varying the current applied to the primary winding includes varying the current according to a delivery profile based on the active agent.

13. The method of claim 8 wherein varying the current applied to the primary winding includes varying the current according to a delivery profile based on at least one parameter indicative of a physical feature of the biological subject.

14. The method of claim 8, further comprising:
storing power to a rechargeable power supply.

15. The method of claim 8, further comprising:
positioning an active electrode and a counter electrode of the iontophoretic delivery device on a biological subject after storing power to the rechargeable power supply before varying the current applied to the primary winding to generate the varying electromagnetic field such that active agent is supplied to the biological entity in response to stored power.

16. A method of forming an inductively powered iontophoretic device, comprising:
forming an inductor element on at least a first substrate having a first surface and a second surface opposing the first surface; and
electrically coupling the inductor element to an iontophoresis device comprising an active electrode assembly and a counter electrode assembly, the active electrode assembly including at least one active agent reservoir and at least one active electrode element operable to provide an electromotive force to drive an active agent from the at least one active agent reservoir, the counter electrode assembly including at least one counter electrode element;
wherein the inductor element is operable to provide a voltage across at least the active and the counter electrode elements in response to a varying electromagnetic field applied to the inductor element from an external source.

17. The method of claim 16 wherein forming an inductor element on at least a first substrate includes depositing a conductive trace on at least the first surface of the first substrate; wherein the conductive trace is operable to provide a voltage across at least the active and the counter electrode elements in response to a varying electromagnetic field applied to the conductive trace.

18. The method of claim 16 wherein forming an inductor element on at least a first substrate includes forming a first portion of the inductor element on the first substrate, and further comprising:
forming a second portion of the inductor element on a second substrate having a first surface and a second surface opposing the first surface.

19. The method of claim 16 wherein forming a first portion of the inductor element on the first substrate and forming a second portion of the inductor element on the second substrate comprises:
depositing a first conductive trace on the first surface of the first substrate;
depositing a second conductive trace on the first surface of the second substrate; and
forming a laminate comprising the first and the at least second substrates.
wherein the first and the second conductive traces are electrically coupled to form a multi-loop inductor, and the electrically coupled first and the second conductive traces are operable to provide a voltage across at least the active and the counter electrode elements in response to a varying electromagnetic field applied to the first and the second conductive traces.

20. The method of claim 16 wherein forming an inductor element on at least a first substrate comprises:
forming a photoresist mask for patterning the conductive trace on the first surface of the substrate; and
etching the conductive trace on the first surface of the substrate.

21. The method of claim 16, further comprising:
electrically coupling a rechargeable power supply in parallel with the inductor element, the rechargeable power supply operable to store power provided by the inductor element in response to an applied varying electromagnetic field.
